(19) United States
(12) Patent Application Publication
(10) Pub. No.: US 2024/0191201 A1
(43) Pub. Date: Jun. 13, 2024

(54) EXTRACELLULAR VESICLES FROM MESENCHYMAL STROMAL CELLS FOR TREATMENT OF DISEASES

(71) Applicant: AVULOTION AB, Hägersten (SE)

(72) Inventors: Karl-Henrik GRINNEMO, Hägersten (SE); Oscar SIMONSON, Uppsala (SE); Sergey RODIN, Stockholm (SE)

(21) Appl. No.: 18/554,581

(22) PCT Filed: Apr. 22, 2022
(86) PCT No.: PCT/EP2022/060665

§ 371 Date: Oct. 9, 2023

(30) Foreign Application Priority Data
Apr. 23, 2021 (SE) 2150516-9

(51) Int. Cl. C12N 5/077; A61K 35/12

(52) U.S. Cl. CPC C12N 5/0669; A61K 35/12; A61K 2035/124; C12N 2533/52

(57) ABSTRACT

The present invention relates to methods for obtaining extracellular vesicles (EVs) from cells such as mesenchymal stromal cells (MSCs), wherein the cells are cultured in the presence of polypeptides from the extracellular matrix proteins laminin alpha-5, laminin alpha-4 or their functional fragments, or in the presence of polypeptides comprising the extracellular domain of human MCAM protein. The invention further relates to EVs obtained by the above methods. The EVs are useful in the treatment and prophylaxis of medical conditions such as inflammatory diseases, ischemic heart disease and acute respiratory distress syndrome.

### TECHNICAL FIELD

The present invention relates to methods for obtaining extracellular vesicles (EVs) from cells such as mesenchymal stromal cells (MSCs), wherein the cells are cultured in the presence of polypeptides from the extracellular matrix proteins laminin alpha-5, laminin alpha-4 or their functional fragments, or in the presence of polypeptides comprising the extracellular domain of human MCAM protein. The invention further relates to extracellular vesicles (EVs) obtained by the above methods. The EVs are useful in the treatment and prophylaxis of medical conditions such as inflammatory diseases, ischemic heart disease and acute respiratory distress syndrome.

### BACKGROUND ART

Extracellular vesicles (EVs) are lipid-membrane enclosed vesicles that are generated by vast majority of cells (1). EVs contain proteins, nucleic acids and lipids and act as important intercellular communicators, which is facilitated by receptors on the membrane of EVs (1, 2). Exosomes, microvesicles and apoptotic bodies represent the major subtypes of EVs. Exosomes are produced inside the cells, released via the endosomal pathway and range between approximately 30 and 100 nm in diameter. Microvesicles are budding from cell plasma membrane and range between approximately 50 and 1000 nm. Apoptotic bodies are released during cell death, contain various parts of the cell and range between approximately 50 and 5000 nm.

Since EVs are able to deliver various molecules to cells in normal and pathological conditions, they are a promising toll for medicine. Thus, EVs can be used as vehicles for delivery of various exogenous molecules into cells (3-5). Also, EVs produced by several types of cells, particularly mesenchymal stromal cells and dendritic cells are tested as medical drugs (6-8).

Mesenchymal stromal cells (MSC) are defined by: (1) expression of certain cell membrane markers (CD73+, CD90+, CD105+); (2) lack of expression of certain markers (CD11b−, CD14−, CD34−, CD45−, CD19−, CD79a−, HLA−DR−); (3) plastic adherence; and (4) trilineage multipotency (ability to differentiate into osteoblasts, chondrocytes and adipocytes) in in vitro and in vivo tests (9). MSCs can be obtained from many tissues and organs of the body such as bone marrow, Wharton's jelly, fat tissue, oral cavity, the heart and teeth (10). Alternatively, the MSCs can be differentiated from stem cells. MSCs have regenerative and immunomodulatory capacities and, hence, are used in preclinical and clinical trials for treatment of various diseases (10). It has been demonstrated that the effect of MSCs is, at least partially, of paracrine nature (11) and EVs produced by MSCs are one of the main paracrine factors. It is hard to compare the regenerative and immunomodulatory effects of MSCs and their EVs, but it has been widely regarded that EVs elicit only partial and weaker effects than the MSCs of their origin (12).

Importantly, the biological effect of EVs produced by MSCs depends on the culture conditions (13). Thus, EVs produced by same MSCs cultured in vitro under different cell culture conditions may have completely different properties, may critically vary in their biological activity from fully inactive to biologically active and should be regarded as different populations of EVs. It is important to find in vitro cell culture conditions that render the cells to produce biologically active EVs.

Living cells should be stored at ultralow temperatures (at temperature of liquid nitrogen) and pre-processed before injection into patients using centrifuges and sterile laminar flow hood. The equipment for the storage and pre-processing of cells is missing in the vast majority of hospitals that imposes a significant storage and logistical problems on the use of MSCs and any other cells in medicine. On the other hand, EVs can be stored using standard freezers and injected into patients directly after thawing without the need for pre-processing. Therefore, biologically active EVs can overcome the storage and logistical problems of cell therapies.

Extracellular matrix (ECM) proteins reside between cells in all organs and tissues and are crucial for homeostasis and pathophysiological processes (14). The ECM does not only provide a mechanical support for the cells, but also provides a necessary signaling for a correct function and phenotype stability of the cells. There are a strong clinical evidences between pathological rearrangement of ECM and adverse medical outcomes particularly in patients with myocardial infarction and heart failure (14). Thus, accumulation of fibrillar collagens such as collagen I and loss of basement membrane collagens such as collagen IV is associated with poor prognosis in patients with heart failure (14). Preventing pathological rearrangement of ECM in diseased organs and tissues is an important medical problem.

Macrophages are key regulators of inflammatory response. Among them, M1 macrophages promote the inflammatory response, while M2 macrophages trigger the resolution of inflammation (15). The M1 and M2 polarized macrophages are different in expression of certain cytokines and cellular receptors. Thus, M2 macrophages are characterized with significantly higher ratio of IL-10 (anti-inflammatory cytokine) to IL-12 expression levels, higher expression of CD-206 and absence of expression of CD-80 in comparison with that in M1 macrophages (16). It has been shown that MSCs are able to convert M1 to M2 phenotypes in in vitro assays and in vivo models of inflammatory diseases (17).

Restoration of organs after injuries is heavily rely on activation of fibroblasts that is a result of external inflammatory stimuli for instance signals from IL-6 or reactive oxygen species (30). Activated fibroblasts are the main source of de novo produced ECM (27). Excessive number of activated fibroblasts may lead to advanced fibrosis that causes excessive stiffening, hostile ECM milieu and, subsequently, loss of function of the organ (27). Thus, advanced fibrosis of the left ventricle may lead to cardiac insufficiency and even heart failure in the patients after myocardial infarction. One important marker of activated fibroblasts is platelet-derived growth factor receptor β (PDGFR-β) (28, 29).

Laminins (LNs) are a major family of basement membrane proteins and are heterotrimeric glycoproteins composed of α, β and γ chains (18). The chains exist in 5, 4, and 3 genetically distinctive types, respectively. They are named according to chain composition, e.g. LN-511 consists of α5, β1, and γ1 chains. Laminins are capable of signaling into cells via interaction with cell membrane receptors and largely affect the function of cells (18). One cell membrane receptor of LNs is MCAM (also known as CD146) that is also capable of homophilic interactions (19, 20). Expression of MCAM correlates with multipotency of MSCs (21, 22).

Laminin E8 fragments are truncated proteins composed of the C-terminal regions of the α, β and γ chains. Laminin E8 fragment can be obtained either by enzymatic digestion of full-length laminin (25) or as recombinant proteins (26). Although significantly smaller in size, laminin E8 fragments preserve a significant part of cellular receptor-binding and signaling activities of full-length laminin molecules (26).

Ma, Y. et al. (31) discloses a method for obtaining EVs comprising culturing NPCs on laminin-coated culture dishes with proliferation medium and isolating EVs from the culture supernatants. However, Ma et al. does not disclose specific laminins such as laminins comprising an α5 chain or an α4 chain.

WO 2017/186273 discloses a method for culturing mesenchymal stem cells (MSCs) under hypoxic conditions in the presence of (i) at least one laminin comprising an α5 chain and/or (ii) at least one laminin comprising an α4 chain. However, there is no disclosure in the prior art that extracellular vesicles, obtained from cells that are cultured in the presence of human laminins or a human MCAM polypeptide, have advantageous effects compared with previously known EVs. Consequently, there is a need for new methods for obtaining EVs that have improved properties, such as improved usefulness in medical treatment and prophylaxis.

### BRIEF DESCRIPTION OF THE DRAWINGS

FIG. 1. Functional recovery of the heart in a model of ischemia reperfusion injury four weeks after the operation. The mice were treated with phosphate buffer (depicted as Control), EVs isolated from MSCs cultured under standard conditions (depicted as EVs) and biologically active EVs isolated from MSCs cultured in the presence of laminin-521 (depicted as baEVs). The function of the hearts has been assessed four weeks after the operation using echocardiography. (A) Left Ventricular Ejection Fraction (LVEF) did not differ in Control and EVs groups, but was significantly (p<0.05) restored in baEVs group. Importantly, mice from baEVs group exhibited normal LVEF indicating recovery of the heart function in the mice treated with the biologically active EVs. (B) Fractional shortening did not differ in Control and EVs groups, but was significantly (p<0.05) restored in baEVs group. Error bars show standard deviation.

FIG. 2. Relative amounts of PDGFR-β mRNA transcripts in the hearts of mice with reperfusion injury treated with various VE preparations or PBS measured using quantitative reverse transcriptase polymerase chain reaction analysis 24 hours after the injection. The EV preparations were isolated from MSCs cultured on plastic (Plastic), laminin-521 (LN-521) or laminin-421 (LN-421). Error bars show standard deviation. ** p<0.01.

FIG. 3. Kaplan-Meier curves for ICU rat model treated with saline buffer (control), biologically active EVs isolated from MSCs cultured in the presence of laminin-521 and laminin-421 (baEVs) and the same MSCs (MSC). Each group contained five animals. The rats treated with baEVs exhibited no mortality during five days after the treatment. Both control and MSC-treated rats demonstrated significant mortality during five days after the treatment.

FIG. 4. Analysis of M1 to M2 macrophage conversion by EVs isolated from MSCs cultured on laminin-521 (LN-521), laminin-421 (LN-421), E8 laminin-511 fragment (E8-511), E8 laminin-411 fragment (E8-411), MCAM chimeric molecule (MCAM), laminin-111 (LN-111) or on plastic (Control). (A) IL-10/IL-12 mRNAs ratios. (B) Percentage of CD-80 positive cells. Error bars show standard deviation.

FIG. 5. Influence of EVs isolated from MSCs cultured on laminin-521 (LN-521), laminin-421 (LN-421), E8 laminin-511 fragment (E8-511), E8 laminin-411 fragment (E8-411), MCAM chimeric molecule (MCAM), laminin-111 (LN-111) or on plastic (Control) on the ability of activated PBMCs to produce anti-inflammatory IL-10 measured using ELISpot assay. Error bars show standard deviation.

### DESCRIPTION OF THE INVENTION

It is an object of the present invention to overcome the above-mentioned problems and provide a source of biologically active EVs for use in medicine.

It has surprisingly been found that EVs isolated from cell culture media conditioned by MSCs cultured in the presence of a laminin containing an α5 or α4 chain, in the presence of MCAM, or in the presence of a combination of these polypeptides (i) rescue the functionality of several organs in animal models of several diseases such as myocardial infarction and critical illness; and (ii) rescue the composition of extracellular matrix (ECM) proteins in the organs. EVs isolated from the same MSCs cultured under standard conditions do not rescue the functionality of the organs and do not affect the ECM. Another unexpected finding is that EVs isolated from MSCs cultured in the presence of the laminins, MCAM, or a combination thereof, exhibit stronger therapeutic effect than the parental MSCs.

Consequently, in a first aspect the invention provides a method for obtaining extracellular vesicles (EVs), said method comprising:
(a) culturing multipotent stem cells, multipotent progenitor cells, or endothelial cells in a cell culture medium, in the presence of a composition comprising at least one polypeptide selected from the group consisting of:
   (i) a polypeptide comprising a human laminin α5 chain or a functional variant thereof;
   (ii) a polypeptide comprising a human laminin α4 chain or a functional variant thereof; and
   (iii) a polypeptide comprising the extracellular domain of human MCAM or a functional variant thereof; and
(b) isolating extracellular vesicles from the cell culture medium.

Preferably, the “multipotent stem cells, multipotent progenitor cells, or endothelial cells” are multipotent stem cells or multipotent progenitor cells.

Preferably, the multipotent stem cells or multipotent progenitor cells are mesenchymal stromal cells (MSCs). The MSCs can be obtained from a source selected from the group consisting of bone marrow, Wharton's jelly, fat tissue, oral cavity, the heart and teeth. For instance, the MSCs are obtained from bone marrow. Alternatively, the MSCs can be differentiated from stem cells or transdifferentiated from somatic cells, including somatic stem cells.

The composition can be present directly in the cell culture medium, or used as a substratum for cell culture. When used as a substratum for cell culture, the composition preferably comprises at least 10% (w/w) of the at least one polypeptide, such as at least 20%, 25%, 30%, 40%, 50% or 100% (w/w).

Preferably, the polypeptides comprising a human laminin α5 or α4 chain, or a functional variant thereof, further comprise a human laminin β chain, such as a β1 or 2 chain, as well as a γ chain, such as a γ1, γ2 or γ3 chain. The β and γ chains form a heterotrimeric laminin structure together with the α chain. When the polypeptide comprises a functional variant, such as a truncated α5 or α4 chain, the β and γ chains are preferably corresponding functional variants, such as truncated β and γ chains. Examples of truncated laminin chains are chains included in laminin E8 fragments.

In one aspect of the invention, the polypeptide comprising a human laminin α5 chain, or a functional variant thereof, is selected from the group consisting of laminin-20, laminin-521, laminin-522, and laminin-523, including E8 fragments thereof, such as laminin E8-511. Preferably, the polypeptide is laminin-521 or laminin E8-511.

Preferably, the polypeptide comprising a human laminin α5 chain, or a functional variant thereof, is selected from the group consisting of (i) a polypeptide comprising the human laminin α5 amino acid sequence shown as SEQ ID NO: 1; (ii) a polypeptide having at least 60% sequence identity, such as at least 70%, 75%, 80%, 85%, 90%, or 95% identity, with SEQ ID NO: 1; and (iii) a polypeptide comprising a fragment of the laminin α5 chain, said fragment shown as positions 2534-3323 in SEQ ID NO: 1.

In another aspect of the invention, the polypeptide comprising a human laminin α4 chain, or a functional variant thereof, is selected from the group consisting of laminin-411, laminin-421, laminin-422, and laminin-423, including E8 fragments thereof, such as laminin E8-411. Preferably, the polypeptide is laminin-421 or laminin E8-411.

Preferably, the polypeptide comprising a human laminin α4 chain, or a functional variant thereof, is selected from the group consisting of (i) a polypeptide comprising the human laminin α4 amino acid sequence shown as SEQ ID NO: 2; (ii) a polypeptide having at least 60% sequence identity, such as at least 70%, 75%, 80%, 85%, 90%, or 95% identity, with SEQ ID NO: 2; and (iii) a polypeptide comprising a fragment of the laminin α4 chain, said fragment shown as positions 636-1456 in SEQ ID NO: 2.

In a further aspect of the invention, the polypeptide comprising the extracellular domain of human MCAM or a functional variant thereof is selected from the group consisting of (i) a polypeptide comprising the amino acid sequence shown as SEQ ID NO: 3 or SEQ ID NO: 4; and (ii) a polypeptide having at least 60% sequence identity, such as at least 70%, 75%, 80%, 85%, 90%, or 95% identity, with SEQ ID NO: 3 or SEQ ID NO: 4. As shown in the Sequence Listing, SEQ ID NO: 3 represents the full-length sequence of human MCAM, while SEQ ID NO: 4 represents the extracellular domain of human MCAM.

Optionally, the polypeptide comprising the extracellular domain of human MCAM, or a functional variant thereof, is fused to a portion, such as an Fc portion, of human IgG1. A suitable Fc portion of human IgG1 may comprise the amino acid sequence shown as SEQ ID NO: 6. The IgG1 polypeptide may be connected to the MCAM polypeptide by a peptide linker, such as the linker shown as SEQ ID NO: 5. The MCAM-Fc fusion protein is preferably in the form of a homodimer wherein each monomer comprises a human MCAM polypeptide, a linker and an Fc portion of human IgG1. A suitable MCAM-Fc fusion protein is commercially available from R&D Systems, Inc. (Catalog No. 9709-MA), and comprises SEQ ID NOS: 4, 5 and 6.

According to the invention, the composition to be used can comprise a mixture of polypeptides such as (i) a mixture of a laminin comprising an α5 chain and a laminin comprising an α4 chain, such as a mixture of laminin-521 and laminin 421; or (ii) a mixture of a polypeptide comprising the extracellular domain of human MCAM and a laminin, or laminins, comprising an α5 chain or an α4 chain.

In the method according to the invention, the extracellular vesicles can be isolated from the cell culture medium by methods known in the art, such as disclosed by Wiklander et al. (13). Suitable methods include e.g. ultracentrifugation, sucrose density ultracentrifugation, differential centrifugation, tangential flow filtering, size exclusion chromatography, and combinations thereof.

In another aspect, the invention provides extracellular vesicles (EVs) obtained by the method as disclosed above. Included in the invention is also a pharmaceutical composition comprising such extracellular vesicles, in combination with at least one pharmaceutically acceptable constituent.

The extracellular vesicles according to the invention are useful for medical purposes, in particular for the treatment or prophylaxis of a medical condition selected from the group consisting of: ischemic and non-ischemic heart failure including heart failure with preserved ejection fraction and heart failure with reduced ejection fraction; heart insufficiency; myocardial infarction; congenital heart disease; myocarditis; valve dysfunction; acute respiratory distress syndrome (ARDS); critical illness myopathy (CIM); ventilator induced diaphragm muscle dysfunction (VIDD); graft-versus-host disease (GvHD); solid organ rejection; rejection of cell or tissue transplants; inflammatory bowel diseases (IBD) such as Crohn's disease and ulcerative colitis; rheumatoid diseases such as arthritis; inflammation-driven or immunologically induced diseases such as multiple sclerosis, ALS, sarcoidosis, idiopathic pulmonary fibrosis, psoriasis, dermatitis, or eczema; allergies such as allergies to food, animals, plants, medicines, chemicals, metals, or dust; autoimmune diseases such pemphigus, type 1 diabetes, systemic lupus erythematosus (SLE), multiple sclerosis (MS), or Guillain-Barre syndrome; diabetes type 2; tumor necrosis factor (TNF) receptor-associated periodic syndrome (TRAPS); deficiency of the interleukin-1 receptor antagonist (DIRA); endometriosis; autoimmune hepatitis; scleroderma; myositis; stroke; acute spinal cord injury; vasculitis; organ failure such as kidney failure, liver failure, lung failure, or heart failure; cancer including lung cancer and skin cancer; and burns including thermal and chemical burns.

The extracellular vesicles according to the invention are particularly useful in the treatment or prophylaxis of a cardiovascular disease, such as ischemia reperfusion injury of the heart, or a respiratory disease, such as acute respiratory distress syndrome (ARDS).

The invention further includes a method for treatment or prophylaxis of a medical condition, said method comprising administering, to a subject in need thereof, a therapeutically effective amount of extracellular vesicles obtained according to the present invention. The medical condition can be any one of those stated above, and include in particular cardiovascular diseases, such as ischemia reperfusion injury of the heart, and respiratory diseases, such as acute respiratory distress syndrome (ARDS).

In a further aspect of the invention, the extracellular vesicles are useful in coating of medical devices such as prostheses or grafts, including prosthetic and biological valves. Included in the invention is also such a medical device, coated with extracellular vesicles obtained by the methods disclosed herein.

### Definitions

“Extracellular vesicles or EVs” are lipid-membrane enclosed vesicles that are generated by vast majority of cells (1). EVs contain proteins, nucleic acids and lipids and act as important intercellular communicators, which is facilitated by receptors on the membrane of EVs (1, 2). Term “extracellular vesicles” includes exosomes, microvesicles and apoptotic bodies that represent the major subtypes of EVs. Exosomes are produced inside the cells, released via the endosomal pathway and range between approximately 30 and 100 nm in diameter. Microvesicles are budding from cell plasma membrane and range between approximately 50 and 1000 nm. Apoptotic bodies are released during cell death, contain various parts of the cell and range between approximately 50 and 5000 nm.

The term “multipotent stem cells” refers to the ability of such cells: (i) to give rise to one or more types of somatic cells (fully differentiated cells) and (ii) their significant proliferation potential. The term “multipotent progenitor cells” refers to multipotent cells that are direct predecessors of somatic cells. The term “multipotent” means the ability to differentiate into discrete cell types or only one cell type of somatic cells. However, unlike stem cells, progenitor cells have a limited proliferation potential.

“Endothelial cells” are cells which create the thin-walled endothelium that lines the inner surface of blood vessels and lymphatic vessels, forming an interface between circulating blood or lymph in the lumen and the rest of the vessel wall.

The term “Mesenchymal stromal cells” or “MSCs” refers to cells that comply with the following definition: (1) expression of certain cell membrane markers CD73, CD90 and CD105; (2) lack of expression of CD11b, CD14, CD34, CD45, CD19, CD79a and HLA-DR; (3) plastic adherence; and (4) trilineage multipotency (ability to differentiate into osteoblasts, chondrocytes and adipocytes) in in vitro and in vivo tests (9). MSCs can be obtained from many, if not all, tissues and organs of the body such as bone marrow, Wharton's jelly, fat tissue, oral cavity, the heart and teeth (10). Alternatively, the MSCs can be differentiated from stem cells or transdifferentiated from other types of cells. For instance, MSCs can be differentiated from human pluripotent cells.

The term “biologically active” or “active” EVs refers to their ability to significantly improve the function of organs and tissues in in vivo models of diseases, particularly inflammatory diseases.

The term “conditioned medium” refers to cell culture medium that has been in contact with cells and contains factors produced by the cells.

The term “polypeptide” or “protein” refers to a polymer of the 20 protein amino acids, or amino acid analogs, regardless of its size or function. Thus, exemplary polypeptides include gene products, naturally occurring or native proteins, homologs, orthologs, paralogs, fragments and other equivalents, variants, and analogs of the foregoing.

The term “substratum” or “substrate” refers to a surface on which an organism (cell) lives. The surface is suitable for culturing cells such as multipotent stem cells, multipotent progenitor cells, and endothelial cells. The substratum can be used to coat supportive surfaces, such as cell culture dishes, beads, porous structures for culturing of cells, microcarriers for bioreactors, internal surfaces of bioreactors, or other surfaces suitable for culturing cells.

According to the present description and claims, a reference to a product or method “comprising” certain features should be interpreted as meaning that it includes those features, but that it does not exclude the presence of other features, as long as they do not render the invention unworkable. In reference to the compounds or compositions according to the invention, the term “consisting essentially of” means that specific further components can be present, namely those not materially affecting the essential characteristics of the compound or composition.

The term “variant” is used herein to refer to an amino acid sequence that is different from the reference protein by one or more amino acids, e.g., one or more amino acid substitutions, inversions or insertions (additions) or deletions. A variant of a reference protein also refers to a variant of a fragment of the reference protein. A variant can also be a “functional variant,” in which the variant retains some or all of the activity of the reference protein as described herein.

The term “fragment,” when used in reference to a protein, refers to a protein in which amino acid residues are deleted as compared to the reference protein itself, but where the remaining amino acid sequence is usually identical to the corresponding positions in the reference protein. Such deletions can occur at the amino-terminus or carboxy-terminus of the reference protein, or alternatively both. A fragment can also be a “functional fragment,” in which case the fragment retains some or all of the activity of the reference protein as described herein. A functional fragment may be a truncated fragment, such as a laminin E8 fragment (see below).

With regard to the polypeptides and compositions according to the invention, the terms “activity”, and “functional” refer to e.g. one or more of the following features: (1) the cells cultured in the presence of the active or functional polypeptides produce EV that are able to prevent pathological rearrangements of ECM in organs and tissues in animal models of inflammatory diseases via inhibition of excessive activation of fibroblasts as described in the Examples of the invention; (2) the cells cultured in the presence of the active or functional polypeptides produce EV that are able to convert M1 to M2 macrophages in in vitro assays as described in the Examples of the invention; (3) the cells cultured in the presence of the active or functional polypeptides produce EV that are able to convert M1 to M2 macrophages in in vivo treatments of inflammatory disorders; (4) the cells cultured in the presence of the active or functional polypeptides produce EV that are able to treat inflammatory disorders in the animal models and patients; (5) the active or functional polypeptides are able to bind cellular receptors on the surface of the cells such as integrins, Dystroglycan receptor, Lutheran receptor and MCAM similarly to the proteins they originate from; and (6) the active or functional polypeptides are able to induce similar signaling inside the cells as the proteins they originate from.

One example of a functional fragment of a laminin is its E8 fragment. The term “laminin E8 fragment” refers to a truncated protein of about 150 kDa composed of the C-terminal regions of the α, β and γ chains. A laminin E8 fragment contains the active integrin-binding site comprising the laminin globular 1-3 domains of the a chain. The globular 1-3 domains are represented by positions 2736-3292 of the laminin 5 chain (SEQ ID NO: 1), and by positions 833-1402 of the laminin α4 chain (SEQ ID NO: 2). Laminin E8 fragments may comprise an α5 chain represented by positions 2534-3323 in SEQ ID NO: 1; or an α4 chain represented by positions 636-1456 in SEQ ID NO: 2.

According to the invention, a pharmaceutical composition may comprise various pharmaceutically acceptable constituents, such as solvents, buffers, carriers, stabilizers, preservatives, etc. The term “pharmaceutically acceptable” means being useful in preparing a pharmaceutical composition that is generally safe, non-toxic and neither biologically nor otherwise undesirable, and includes being useful for veterinary use as well as human pharmaceutical use.

### EXPERIMENTAL METHODS

### Cell Culture Substrata

Human recombinant laminins were purchased from BioLamina AB (Sweden). Human recombinant E8 laminin molecules were purchased from AMSBIO (U.K.).

A recombinant human MCAM Fc chimera was purchased from R&D Systems, Inc (Catalog No. 9709-MA). The chimera is a disulfide-linked homodimer wherein each monomer comprises (i) human MCAM (Val24-Gly559; SEQ ID NO: 4); (ii) the peptide linker IEGRMD (SEQ ID NO: 5); and (iii) a human IgG1 Fc portion (SEQ ID NO: 6).

### Cell Culture Dish Coating

Laminin coating: Tissue cell culture plates from TPP (Switzerland) were coated overnight at +4° C. with sterile solutions of laminins, such as human recombinant laminin-521, laminin-421 or laminin-111, all at a concentration of 10 μg/ml (1.5 μg/cm²) in phosphate buffered saline (PBS). In case when a mixture of two laminins was used, they were taken at equal weight to weight ratio at a concertation of 0.75 μg/cm² each.

Laminin E8 fragment coating: Tissue cell culture plates from TPP (Switzerland) were coated overnight at +4° C. with sterile solutions of laminin E8 fragments, such as E8 laminin-511 and E8 laminin-411, at a concentration of 1.5 μg/cm² in PBS.

MCAM coating: 25 cm² cell culture treated flasks from TPP (Switzerland) were coated overnight at +4° C. with sterile solutions of the recombinant human MCAM chimeric molecule at a concentration of 4.2 g/ml (0.5 μg/cm²) in phosphate buffered saline (PBS).

In the following Examples of the invention, the term “MCAM chimeric molecule” refers to the MCAM-Fc fusion protein comprising SEQ ID NOS: 4, 5 and 6, purchased from R&D Systems, Inc. (Catalog No. 9709-MA).

Before use, the flasks were incubated at +37° C. for one hour and washed twice with PBS. Prewarmed cell culture medium was then added.

### Culturing of MSCs

Bone marrow derived MSCs (BM-MSCs) were cultured on cell culture treated flasks with and without the coatings in Dulbecco's Modified Eagle's Medium (DMEM) with low glucose (Life Technologies, USA) supplemented with 10% of bovine serum (Thermo Fisher Scientific, USA) or in StemMACS™ MSC Expansion Medium (Miltenyi Biotec). For passaging, the cells were washed once with phosphate buffered saline (PBS) and removed from the flasks by exposure to TrypLE Express (GIBCO, Thermo Fischer, USA) for approximately 5 minutes. Culturing medium was next added to inhibit TrypLE Express, the cell suspension was centrifuged for 5 minutes at 180×g at room temperature and the supernatant was discarded. After that, the cells were resuspended in prewarmed culture medium, counted and plated at approximately 6 000 cells/cm². All cultures were done in humidified cell culture incubators at +37° ° C. in 5% CO2.

Isolation of Extracellular Vesicles from Cultured Cells

The cells were cultured as described above until 90% confluency. After that, the medium was changed to Opti-MEM medium (ThermoFisher, US) without serum and the cells were incubated for additional 48 hours in a humidified cell culture incubator at +37° C. in 5% CO2. The medium (conditioned medium) was collected, centrifuged for 10 minutes at 120×g to remove the floating cells, and then for additional 10 minutes at 300×g to remove cellular debris. After that, the medium was filtered using 0.2 μm filter, EVs were collected using ultracentrifugation at 110,000×g for 1 hour and solubilized in a small volume of phosphate-buffered saline (PBS). The preparation of EVs were analyzed using a NanoSight NS500 device to determine the EV size distribution and concentration.

Modelling of Ischemia Reperfusion Injury in Mice and Treatment with EVs

All animal experiments were carried out according to the guidelines of the Swedish Board of Agriculture and approved by the ethical committee. The mouse was under general anesthesia with Isoflurane, orally intubated and ventilated by a ventilator system designed for rodents. The heart was exposed through a left thoracotomy, pericardium opened and Left Anterior Descending artery (LAD) was next ligated (7-0 polypropylene suture) over a thin tube for 40 minutes after which the tube was removed and with that the blood flow restored. The heart was reperfused and EVs (or control solution) were injected into the previously ischemic area in a total volume of up to 30 μl. All injections were performed in a blinded fashion. The thoracotomy was closed by 6-0 polypropylene single sutures closing layer by layer. The inhalation anesthesia was stopped after which the mouse woke up and was followed for the first hours. All procedures were performed a blinded fashion. For analysis of Pdgfrb gene expression, the mice were sacrificed 24 hours after the injection and the whole hearts were obtained for subsequent RNA isolation (see below Quantification of mRNAs).

### Echocardiography

For long-term experiment, the mice were undergoing echocardiography under general anesthesia (Isoflurane) the day before surgery, the first day after surgery, and at 14 and 28 days after surgery. At these timepoints, the regional and global function of the left ventricle was studied and compared to baseline (day −1). All analyses were performed in a blinded fashion.

### Intensive Care Unit (ICI) Rat Model

Adult female Sprague-Dawley rats were exposed to controlled mechanical ventilation, neuromuscular blockade and deep sedation for five days (23, 24). All rats were mechanically ventilated, sedated with isoflurane (maintained at minimum alveolar concentration <0.5% and adjusted to maintain hemodynamic stability), and pharmacologically paralyzed postsynaptically with cobratoxin (maintained by continuous infusion; 187 mg/day). Protein and fluid balance were maintained in all experimental animals throughout the duration of mechanical ventilation. At the end of the experimental period of five days or if the rats deteriorated, the animals were euthanized.

Monocyte-Derived Macrophage Isolation and Differentiation into M1 Polarization

Buffy coats from male healthy donors were obtained at the Blood Donor Center, Uppsala. Human peripheral blood mononuclear cells (PBMCs) were isolated from buffy coats using BD Vacutainer® CPT™ Tubes (BD Biosciences, US). To isolate monocytes, the cells were sorted by magnetic activated cell sorting (MACS) using magnetic beads conjugated with anti-human CD14 (Miltenyi Biotech). To acquire non-polarized macrophages, the monocytes were cultured for 6 days in RPMI 1640 culture medium (Life Technologies, USA) supplemented with 10% fetal bovine serum (Thermo Fisher Scientific, USA), 5% human serum (Sigma-Aldrich), 100 units/mL penicillin, 100 mg/mL streptomycin (Thermo Fisher Scientific, USA), 2 mM L-glutamine (Thermo Fisher Scientific, USA) and 25 ng/ml M-CSF (R&D Systems, UK). To achieve M1 polarization, the non-polarized macrophages were additionally supplemented with 10 ng/ml interferon-γ (R&D Systems, UK) and 100 ng/ml lipopolysaccharides from E. Coli (Sigma-Aldrich) for 48 hours. All cultures were done in humidified cell culture incubators at +37° C. in 5% CO2.

### Conversion of M1 to M2 Macrophages by EV's Assay

For each experiment, 5×10⁵ PBMCs were differentiated into M1 macrophages as described above. To test the efficacy of converting M1 to M2 macrophages, 1×10⁹ of EVs were added to the cells and cultured for additional three days. After that, the cells were subdivided into two equal parts. One of them was analyzed using FACS for expression of CD-80 that is a marker of M1 macrophages and another was used to prepare mRNA for subsequent analysis of expression of IL-10 and IL-12 mRNAs. All the experiments were done in triplicates.

### ELISpot Assay for IL-10

PBMCs producing IL-10 were detected using an enzyme-linked immunospot (ELISpot) kit (R&D Systems, Minneapolis, MN, USA) according to the manufacturer's instructions. In brief, 10 million of PBMCs were resuspended in RPMI medium supplemented with 5% of FBS at concentration of 2 million cells/ml and activated by mixing with 10 million PBMCs from another donor, which had been preirradiated with 25 Gy, in the presence of 4×10⁹ of EVs from various preparations. The cells were added to the 96-well plated precoated with antibodies against IL-10 provided with the kit (100 μl per well) and incubated for 24 hours. After that, the cells were washed away, biotinylated antibodies were added and the plates were incubated overnight at +4° C. The signal was visualized using 5-bromo-4-chlor-3-indolyl-phosphate/nitro blue tetrazolium (BCIP/NBT) and the colonies were counted in each well. All experiments were performed in quadruplicates.

### FACS Analysis

The cells were resuspended in ice-cold FACS buffer (2% fetal bovine serum, 0.1% sodium azide in Hank's buffer) and stained with antibody against CD-80 (Thermo Fisher Scientific, USA) labeled with PE fluorophore performed for one hour on ice in the dark. Then, the cells were washed four times with ice-cold FACS buffer and analyzed on FACSCalibur Flow Cytometer (Becton Dickinson). Control cells were incubated with isotypic control antibodies also labelled with PE. The data were analyzed and the proportion of CD-80 positive cells was determined with the CellQuest software (Becton Dickinson).

Quantification of mRNAs

Total RNA from cells or whole mouse hearts was isolated using RNAeasy Microprep kit (Qiagen, USA) according to the manufacturer's instructions. cDNA was synthesized with 0.2 μg of total RNA in 20 μL reaction mixture using High Capacity RNA-to-cDNA kit (Thermo Fisher Scientific, USA) according to the manufacturer's instructions. Quantitative real-time PCR (qRT-PCR) Taqman assays for expression of IL-10 and IL-12 mRNAs were performed using CFX Connect™ Real-Time PCR Detection System (Bio-Rad, USA). All reactions were done in quadruplicates with the use of a pre-developed gene expression assay mix (Applied Biosystems, USA) containing primers and a probe for the messenger RNA of interest. Additionally, each experiment included the assay mix for GAPDH for normalization of the RNA input. All data were analyzed using CFX manager version 3.0 (Bio-Rad).

### Statistics

Statistical significance was determined the by Student's two-tailed t-test for unequal variances.

### EXAMPLES OF THE INVENTION

### Example 1. Extracellular Vesicles (EVs) Isolated from Human Bone Marrow Mesenchymal Stromal Cells for Treatment of Mouse Ischemia Reperfusion Injury of the Heart

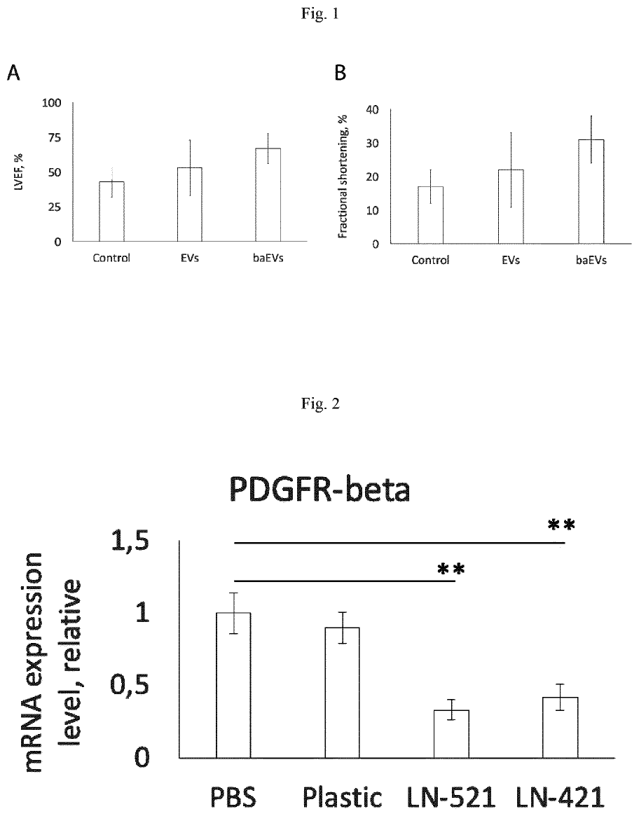

Bone marrow mesenchymal stromal cells that had undergone the same number of divisions were cultured: (1) in standard conditions on plastic in DMEM with low glucose supplemented with 10% of bovine serum to acquire a preparation of standard EVs; and (2) on laminin-521 in StemMACS™ MSC Expansion Medium to acquire a preparation of biologically active EVs (baEVs). Both EVs and baEVs were isolated as described above under “Experimental Methods”. To compare the therapeutic potential, twenty-four mice with ischemia reperfusion injury of the heart were divided into three equal groups: (1) control group treated with PBS (eight animals), (2) EV group treated with 4×10⁹ EV isolated from standard cultures of BM-MSCs, and (3) baEV group treated with 4×10⁹ baEV isolated from BM-MSCs cultured on the laminins. All the treatments were performed as described above. Four weeks after the treatment, control mice exhibited significantly reduced Left Ventricular Ejection Fraction (LVEF) and Fractional shortening indicating significantly reduced functionality of the heart (FIGS. 1A and 1B). The mice treated with standard EVs showed no statistically significant differences with the control group. In contrast, the mice treated with biologically active EVs exhibited significantly (p<0.05) higher LVEF and Fraction shortening with both parameters in the range for normal healthy hearts suggesting that baEVs preserved functionality of the heart after ischemia reperfusion injury.

Since pathological rearrangement of ECM is a major mechanism of heart failure and activated fibroblasts are the main source of de novo produced ECM (cf. the section “Background Art”), expression levels of the marker of activated fibroblasts PBGFR-β were compared in the hearts of mice with reperfusion injury treated with various preparations of EVs and with PBS 24 hours after the injection (FIG. 2). Each experimental group contained four mice with reperfusion injury. The groups were treated with (1) PBS; (2) 4×10⁹ EVs isolated from BM-MSCs cultured on plastic in StemMACS™ MSC Expansion Medium; (3) 4×10⁹ EVs isolated from BM-MSCs cultured on laminin-521 in StemMACS™ MSC Expansion Medium; and (4) 4×10⁹ EVs isolated from BM-MSCs cultured on laminin-421 in StemMACS™ MSC Expansion Medium. The treatment with EVs produced from the cells cultured on laminin-521 or laminin-421 significantly decreased the level of expression of the marker of activated fibroblasts PBGFR-β in comparison with the treatment with PBS, while the treatment with the EVs produced from the cells cultured on plastic exhibited no such effect (FIG. 2).

### Example 2. Therapeutic Effect of Biologically Active EVs and their Parental BM-MSCs in the ICU Rat Model

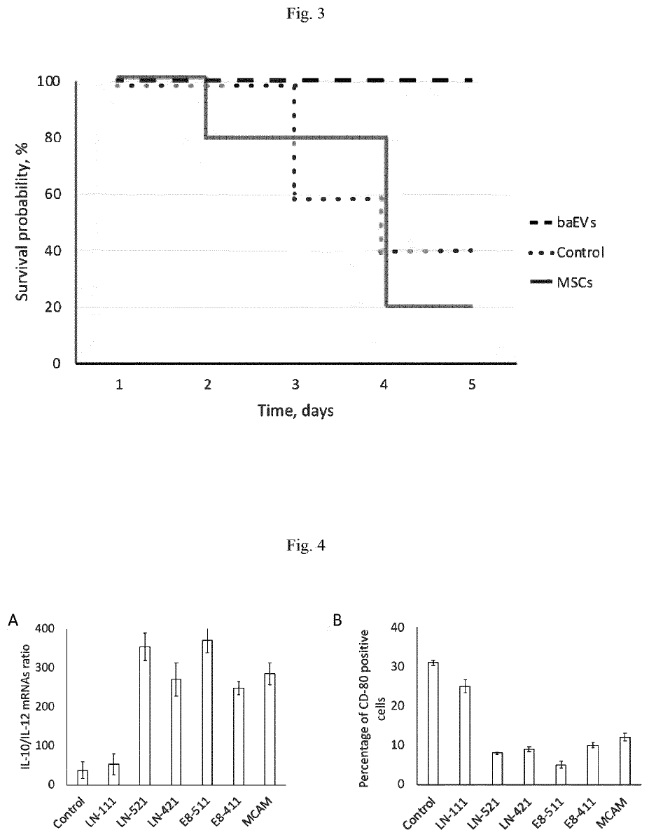

Bone marrow MSCs were cultured on a mixture of laminin-521 and laminin-421 in DMEM with low glucose supplemented with 10% of bovine serum, as described above under “Experimental Methods”. The MSCs were (1) used to isolate biologically active EVs for treatment of the ICU rat model, and (2) used directly for treatment of the ICU rat model which is relevant to human ARDS and ventilator-induced diaphragm dysfunction (VIDD); cf. Dworkin et al. (23). Fifteen rats were used for induction of the ICU rat model and subdivided into three groups containing five animals each. Control, baEVs and MSC groups were treated at the beginning of the experiment with intravenous administration of 1 ml of saline buffer, 3.6×10⁹ baEVs and 0.5 million MSCs, respectively. Both the EVs and MSCs were resuspended in 1 ml of saline buffer. According to the EV concentration measurements, 0.5 million MSCs produced approximately 4×10⁹ EVs and, therefore, the therapeutic effects of these numbers of the cells and the EVs could be directly compared. All rats in the baEV group survived until the end of the experiment, while both Control and MSCs groups exhibited significantly higher mortality (FIG. 3) indicating significantly higher therapeutic effect of baEVs in comparison with that of the parental cells.

### Example 3: Activity of EVs Isolated from BM-MSCs Cultured on Laminin-521, Laminin-421, E8 Laminin-511 Fragment, E8-Laminin-411 Fragment or MCAM Chimeric Molecule in Comparison with that of EVs Isolated from MSCs Cultured on Plastic and Laminin-111 in In Vitro Immunoassays

EV preparations were isolated from MSCs cultured on laminin-521, laminin-421, E8 laminin-511 fragment, E8 laminin-411 fragment, MCAM chimeric molecule, laminin-111 or on plastic as described above under “Experimental Methods”. To compare their biological activity, M1 to M2 conversion assays were performed using EVs from the five EV preparations as described above under Experimental Methods (“Conversion of M1 to M2 macrophages by EVs assay”) and analyzed using FACS and qRT-PCR as described above under Experimental Methods. Macrophages treated with EVs isolated from MSCs cultured on laminin-521, laminin-421, E8 laminin-511 fragment, E8 laminin-411 or MCAM chimeric molecule exhibited significantly (p<0.01) higher IL-10/IL-12 mRNAs ratios that is characteristic for M2 macrophages and significantly (p<0.01) lower percentage of CD-80 positive cells that is a marker of M1 macrophages than the cells treated with EVs produced by MSCs cultured on LN-111 or on plastic (control) (FIGS. 4A and 4B). The increased IL-10/IL-12 mRNAs ratio indicates significantly higher presence of M2 macrophages and the decreased percentage of CD-80 cells indicates significantly lower presence of M1 showing significantly higher M1 to M2 conversion by EVs isolated from MSCs cultured on laminin-521, laminin-421, E8 laminin-511 fragment, E8 laminin-411 or MCAM chimeric molecule in comparison with that of EVs isolated from same MSCs cultured in standard conditions or on laminin-111.

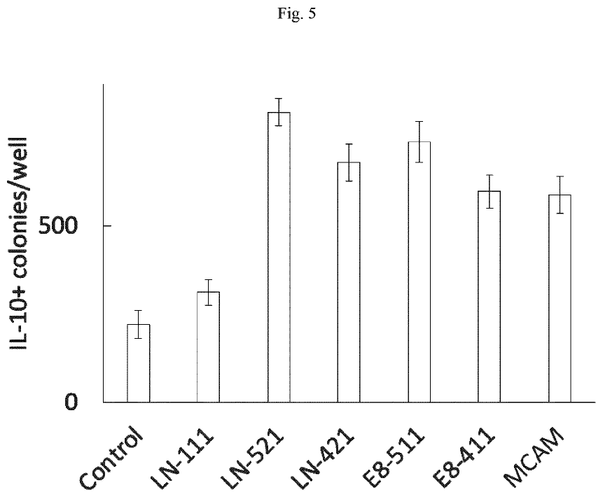

To additionally study the activity of the EV preparations in in vitro immunoassays, we performed IL-10 ELISpot assay on activated human PBMCs with addition of the EV preparations as described above under Experimental Methods (“ELISpot assay for IL-10”). Activated PBMCs treated with EVs isolated from MSCs cultured on laminin-521, laminin-421, E8 laminin-511 fragment, E8 laminin-411 or MCAM chimeric molecule exhibited significantly (p<0.01) higher number of IL-10 colonies that is an anti-inflammatory cytokine than the cells treated with EVs produced by MSCs cultured on LN-111 or on plastic (control) (FIG. 5).

Conclusion: EVs isolated from MSCs cultured on laminin-521, laminin-421, E8 laminin-511 fragment, E8 laminin-411 or MCAM should regarded as a different population from EVs produced from MSCs grown on plastic (control EVs) for the following reasons:
They normalize function of organs in in vivo assays while control EVs are not able to do that.
They inhibit excessive activation of fibroblasts thus impeding pathological rearrangement of ECM in the organs after injury while control EVs are not able to do that.
They significantly stronger affect the key regulators of the inflammatory response in in vitro assays.

### REFERENCES

1. Théry C, Zitvogel L, Amigorena S. Exosomes: Composition, biogenesis and function. Nat Rev Immunol 2002; 2:569-579. doi: 10.1038/nri855.
2. Valadi H, Ekström K, Bossios A, Sjöstrand M, Lee J J, Lötvall J O. Exosome-mediated transfer of mRNAs and microRNAs is a novel mechanism of genetic exchange between cells. Nat Cell Biol 2007; 9:654-659. doi: 10.1038/ncb1596.
3. Alvarez-Erviti L, Seow Y, Yin H, Betts C, Lakhal S, Wood M J A. Delivery of siRNA to the mouse brain by systemic injection of targeted exosomes. Nat Biotechnol 2011; 29:341-345. doi:10.1038/nbt.1807.
4. Pascucci L, Coccè V, Bonomi A, Ami D, Ceccarelli P, Ciusani E, et al. Paclitaxel is incorporated by mesenchymal stromal cells and released in exosomes that inhibit in vitro tumor growth: A new approach for drug delivery. J Control Release 2014; 192:262-270. doi:10.1016/j.jconrel.2014.07.042.
5. Zhang Y, Liu D, Chen X, Li J, Li L, Bian Z, et al. Secreted Monocytic miR-150 Enhances Targeted Endothelial Cell Migration. Mol Cell 2010; 39:133-144. doi:10.1016/j.molcel.2010.06.010.
6. Kordelas L, Rebmann V, Ludwig A-K, Radtke S, Ruesing J, Doeppner T R, et al. MSC-derived exosomes: a novel tool to treat therapy-refractory graft-versus-host disease. Leukemia 2014; 28:970-973. doi:10.1038/leu.2014.41.
7. Nassar W, El-Ansary M, Sabry D, Mostafa M A, Fayad T, Kotb E, et al. Umbilical cord mesenchymal stem cells derived extracellular vesicles can safely ameliorate the progression of chronic kidney diseases. Biomater Res 2016; 20:1-11. doi: 10.1186/s40824-016-0068-0.
8. Besse B, Charrier M, Lapierre V, Dansin E, Lantz O, Planchard D, et al. Dendritic cell-derived exosomes as maintenance immunotherapy after first line chemotherapy in NSCLC. Oncoimmunology 2016; 5:e1071008. doi: 10.1080/2162402X.2015.1071008.
9. Dominici M, Le Blanc K, Mueller I, Slaper-Cortenbach I, Marini F., Krause D S, et al. Minimal criteria for defining multipotent mesenchymal stromal cells. The International Society for Cellular Therapy position statement. Cytotherapy 2006; 8:315-317. doi:10.1080/14653240600855905.
10. Uccelli A, Moretta L, Pistoia V. Mesenchymal stem cells in health and disease. Nat Rev Immunol 2008; 8:726-736. doi:10.1038/nri2395.
11. Grinnemo K H, Löfling M, Nathanson L, Baumgartner R, Ketelhuth D F J, Beljanski V, et al. Immunomodulatory effects of interferon-yon human fetal cardiac mesenchymal stromal cells. Stem Cell Res Ther 2019; 10:371. doi:10.1186/s13287-019-1489-1.
12. Xie H, Wang Z, Zhang L, Lei Q, Zhao A, Wang H, et al. Extracellular Vesicle-functionalized Decalcified Bone Matrix Scaffolds with Enhanced Pro-angiogenic and Pro-bone Regeneration Activities. Sci Rep 2017; 7:1-13. doi:10.1038/srep45622.
13. Wiklander O P B, Brennan M, Lotvall J, Breakefield X O, Andaloussi S E L. Advances in therapeutic applications of extracellular vesicles. Sci Transl Med 2019; 11. doi: 10.1126/scitranslmed.aav8521.
14. Frangogiannis N G. The extracellular matrix in ischemic and nonischemic heart failure. Circ Res 2019; 125: 117-146. doi: 10.1161/CIRCRESAHA.119.311148.
15. Mills C D, Kincaid K, Alt J M, Heilman M J, Hill A M. M-1/M-2 Macrophages and the Th1/Th2 Paradigm. J Immunol 2000; 164:6166-6173. doi: 10.4049/jimmunol.164.12.6166.
16. Bertani F R, Mozetic P, Fioramonti M, Iuliani M, Ribelli G, Pantano F, et al. Classification of M1/M2-polarized human macrophages by label-free hyperspectral reflectance confocal microscopy and multivariate analysis. Sci Rep 2017; 7:1-9. doi: 10.1038/s41598-017-08121-8.
17. Jin L, Deng Z, Zhang J, Yang C, Liu J, Han W, et al. Mesenchymal stem cells promote type 2 macrophage polarization to ameliorate the myocardial injury caused by diabetic cardiomyopathy. J Transl Med 2019; 17:251. doi: 10.1186/s12967-019-1999-8.
18. Domogatskaya A, Rodin S, Tryggvason K. Functional Diversity of Laminins. Annu Rev Cell Dev Biol 2012; 28:523-553. doi: 10.1146/annurev-cellbio-101011-155750.
19. Ishikawa T, Wondimu Z, Oikawa Y, Gentilcore G, Kiessling R, Egyhazi Brage S, et al. Laminins 411 and 421 differentially promote tumor cell migration via α6β1 integrin and MCAM (CD146). Matrix Biol 2014; 38:69-83. doi:10.1016/J.MATBIO.2014.06.002.
20. Flanagan K, Fitzgerald K, Baker J, Regnstrom K, Gardai S, Bard F, et al. Laminin-411 Is a Vascular Ligand for MCAM and Facilitates TH17 Cell Entry into the CNS. PLOS One 2012; 7:e40443. doi: 10.1371/journal.pone.0040443.
21. Sacchetti B, Funari A, Michienzi S, Di Cesare S, Piersanti S, Saggio I, et al. Self-Renewing Osteoprogenitors in Bone Marrow Sinusoids Can Organize a Hematopoietic Microenvironment. Cell 2007; 131:324-336. doi: 10.1016/j.cell.2007.08.025.
22. Russell K C, Phinney D G, Lacey M R, Barrilleaux B L, Meyertholen K E, O'Connor K C. In Vitro High-Capacity Assay to Quantify the Clonal Heterogeneity in Trilineage Potential of Mesenchymal Stem Cells Reveals a Complex Hierarchy of Lineage Commitment. Stem Cells 2010; 28:788-798. doi: 10.1002/stem.312.
23. Dworkin B R, Dworkin S. Learning of Physiological Responses: I. Habituation, Sensitization, and Classical Conditioning. Behav Neurosci 1990; 104:298-319. doi: 10.1037/0735-7044.104.2.298.
24. Dworkin B R, Dworkin S. Baroreflexes of the rat. III. Open-loop gain and electroencephalographic arousal. Am J Physiol—Regul Integr Comp Physiol 2004; 286. doi: 10.1152/ajpregu.00469.2003.
25. Goodman S L. Alpha 6 beta 1 integrin and laminin E8: an increasingly complex simple story. Kidney Int. 1992; 41(3):650-656. doi:10.1038/ki.1992.100
26. Miyazaki T, Futaki S, Suemori H, et al. Laminin E8 fragments support efficient adhesion and expansion of dissociated human pluripotent stem cells [published correction appears in Nat Commun. 2013; 4:1931]. Nat Commun. 2012; 3:1236. doi:10.1038/ncomms2231
27. Cakir S N, Whitehead K M, Hendricks H K L, de Castro Brás L E. Novel Techniques Targeting Fibroblasts after Ischemic Heart Injury. Cells. 2022; 11(3):402. website: doi.org/10.3390/cells11030402
28. Rajkumar V S, Shiwen X, Bostrom M, et al. Platelet-derived growth factor-beta receptor activation is essential for fibroblast and pericyte recruitment during cutaneous wound healing. Am J Pathol. 2006; 169(6):2254-2265. doi: 10.2353/ajpath.2006.060196
29. Donovan, J., Shiwen, X., Norman, J. et al. Platelet-derived growth factor alpha and beta receptors have overlapping functional activities towards fibroblasts. Fibrogenesis Tissue Repair 6, 10 (2013). website: doi.org/10.1186/1755-1536-6-10
30. Ma Y, Iyer R P, Jung M, Czubryt M P, Lindsey M L. Cardiac Fibroblast Activation Post-Myocardial Infarction: Current Knowledge Gaps. Trends Pharmacol Sci. 2017; 38(5):448-458. doi: 10.1016/j.tips.2017.03.001

31 Ma Y, Wang K, Pan J, et al. Induced neural progenitor cells abundantly secrete extracellular vesicles and promote the proliferation of neural progenitors via extracellular signal-regulated kinase pathways. Neurobiol Dis. 2019; 124:322-334. doi:10.1016/j.nbd.2018.12.003

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “NOVI-69179-Sequence-Listing_ST25.txt”, created Oct. 2, 2023, file size 61,440 bytes, is hereby incorporated by reference.

1. A method for obtaining extracellular vesicles (EVs), comprising:
(a) culturing multipotent stem cells, multipotent progenitor cells, or endothelial cells in a cell culture medium, in the presence of a composition comprising at least one polypeptide selected from the group consisting of:
(i) a polypeptide comprising a human laminin α5 chain or a functional variant thereof;
(ii) a polypeptide comprising a human laminin α4 chain or a functional variant thereof; and
(iii) a polypeptide comprising the extracellular domain of human MCAM or a functional variant thereof; and
(b) isolating extracellular vesicles from the cell culture medium.

2. The method according to claim 1, wherein the multipotent stem cells or multipotent progenitor cells are mesenchymal stromal cells (MSCs).

3. The method according to claim 2, wherein the MSCs are obtained from a source selected from the group consisting of bone marrow, Wharton's jelly, fat tissue, oral cavity, heart, and teeth.

4. The method according to claim 2, wherein the MSCs are differentiated from stem cells or transdifferentiated from somatic cells.

5. The method according to claim 1, wherein the composition is used as a substratum for cell culture and comprises at least 10% (w/w) of the at least one polypeptide.

6. The method according to claim 1, wherein the polypeptide comprising a human laminin α5 chain, or a functional variant thereof, is selected from the group consisting of laminin-511, laminin-521, laminin-522, and laminin-523, including E8 fragments thereof.

7. The method according to claim 6, wherein the polypeptide comprising a human laminin α5 chain, or a functional variant thereof, is laminin-521 or laminin E8-511.

8. The method according to claim 1, wherein the polypeptide comprising a human laminin α5 chain, or a functional variant thereof, is selected from the group consisting of:
(i) a polypeptide comprising the laminin α5 chain amino acid sequence shown as SEQ ID NO: 1;
(ii) a polypeptide having at least 60% sequence identity with SEQ ID NO: 1; and
(iii) a polypeptide comprising a fragment of the laminin α5 chain shown as positions 2534-3323 in SEQ ID NO: 1.

9. The method according to claim 1, wherein the polypeptide comprising a human laminin α4 chain, or a functional variant thereof, is selected from the group consisting of laminin-411, laminin-421, laminin-422, and laminin-423, including E8 fragments thereof.

10. The method according to claim 9, wherein the polypeptide comprising a human laminin α4 chain, or a functional variant thereof, is laminin-421 or laminin E8-411.

11. The method according to claim 1, wherein the polypeptide comprising a human laminin α4 chain, or a functional variant thereof, is selected from the group consisting of:
(i) a polypeptide comprising the laminin α4 chain amino acid sequence shown as SEQ ID NO: 2;
(ii) a polypeptide having at least 60% sequence identity with SEQ ID NO: 2; and
(iii) a polypeptide comprising a fragment of the laminin α4 chain shown as positions 636-1456 in SEQ ID NO: 2.

12. The method according to claim 1, wherein the polypeptide comprising the extracellular domain of human MCAM or a functional variant thereof is selected from the group consisting of:
(i) a polypeptide comprising the amino acid sequence shown as SEQ ID NO: 3 or SEQ ID NO: 4; and
(ii) a polypeptide having at least 60% sequence identity with SEQ ID NO: 3 or SEQ ID NO: 4.

13. The method according to claim 1, wherein the composition is used to coat a support chosen from the group consisting of cell culture dishes, beads, microcarriers for bioreactors, and internal surfaces of bioreactors.

14. The method according to claim 1, wherein the extracellular vesicles are isolated from the cell culture medium by a method comprising ultracentrifugation, sucrose density ultracentrifugation, differential centrifugation, tangential flow filtering, size exclusion chromatography, or a combination of thereof.

15. Extracellular vesicles obtained by the method according to claim 1.

16. A pharmaceutical composition comprising extracellular vesicles according to claim 15, in combination with at least one pharmaceutically acceptable constituent.

17-22. (canceled)

23. A medical device coated with extracellular vesicles according to claim 15.

24. The medical device according to claim 23, wherein the medical device is selected from the group consisting of a prosthesis, a graft, a prosthetic valve, and a biological valve.

25. A method for treatment or prophylaxis of a medical condition in a subject in need thereof, the method comprising administering to the subject a therapeutically acceptable amount of the extracellular vesicles according to claim 15.

26. The method for treatment or prophylaxis according to claim 24, wherein the medical condition is selected from the group consisting of ischemic heart failure; non-ischemic heart failure; heart failure with preserved ejection fraction; heart failure with reduced ejection fraction; heart insufficiency; myocardial infarction; congenital heart disease; myocarditis; valve dysfunction; acute respiratory distress syndrome (ARDS); critical illness myopathy (CIM); ventilator induced diaphragm muscle dysfunction (VIDD); graft-versus-host disease (GvHD); solid organ rejection; rejection of a cell or tissue transplant; inflammatory bowel disease (IBD); Crohn's disease; ulcerative colitis; rheumatoid disease; arthritis; inflammation-driven or immunologically induced disease; multiple sclerosis; ALS; sarcoidosis; idiopathic pulmonary fibrosis; psoriasis; dermatitis; eczema; allergies; allergies to food, animals, plants, medicines, chemicals, metals, or dust; autoimmune disease; pemphigus; type 1 diabetes; systemic lupus erythematosus (SLE); multiple sclerosis (MS); Guillain-Barre syndrome; diabetes type 2; tumor necrosis factor (TNF) receptor-associated periodic syndrome (TRAPS); deficiency of the interleukin-1 receptor antagonist (DIRA); endometriosis; autoimmune hepatitis; scleroderma; myositis; stroke; acute spinal cord injury; vasculitis; organ failure; kidney failure; liver failure; lung failure; heart failure; cancer; lung cancer; skin cancer; burns; thermal burns; and chemical burns.


SEQUENCE LISTING (6 sequences)

SEQ ID NO: 1 - protein, 3695 aa, Homo sapiens
MAKRLCAGSALCVRGPRGPAPLLLVGLALLGAARAREEAGGGFSLHPPYFNLAEGARIAASATCGEEAPARGSPRPTEDLYCKLVGGPVAGGDPNQTIRGQYCDICTAANSNKAHPASNAIDGTERWWQSPPLSRGLEYNEVNVTLDLGQVFHVAYVLIKFANSPRPDLWVLERSMDFGRTYQPWQFFASSKRDCLERFGPQTLERITRDDAAICTTEYSRIVPLENGEIVVSLVNGRPGAMNFSYSPLLREFTKATNVRLRFLRTNTLLGHLMGKALRDPTVTRRYYYSIKDISIGGRCVCHGHADACDAKDPTDPFRLQCTCQHNTCGGTCDRCCPGFNQQPWKPATANSANECQSCNCYGHATDCYYDPEVDRRRASQSLDGTYQGGGVCIDCQHHTTGVNCERCLPGFYRSPNHPLDSPHVCRRCNCESDFTDGTCEDLTGRCYCRPNFSGERCDVCAEGFTGFPSCYPTPSSSNDTREQVLPAGQIVNCDCSAAGTQGNACRKDPRVGRCLCKPNFQGTHCELCAPGFYGPGCQPCQCSSPGVADDRCDPDTGQCRCRVGFEGATCDRCAPGYFHFPLCQLCGCSPAGTLPEGCDEAGRCLCQPEFAGPHCDRCRPGYHGFPNCQACTCDPRGALDQLCGAGGLCRCRPGYTGTACQECSPGFHGFPSCVPCHCSAEGSLHAACDPRSGQCSCRPRVTGLRCDTCVPGAYNFPYCEAGSCHPAGLAPVDPALPEAQVPCMCRAHVEGPSCDRCKPGFWGLSPSNPEGCTRCSCDLRGTLGGVAECQPGTGQCFCKPHVCGQACASCKDGFFGLDQADYFGCRSCRCDIGGALGQSCEPRTGVCRCRPNTQGPTCSEPARDHYLPDLHHLRLELEEAATPEGHAVRFGFNPLEFENFSWRGYAQMAPVQPRIVARLNLTSPDLFWLVFRYVNRGAMSVSGRVSVREEGRSATCANCTAQSQPVAFPPSTEPAFITVPQRGFGEPFVLNPGTWALRVEAEGVLLDYVVLLPSAYYEAALLQLRVTEACTYRPSAQQSGDNCLLYTHLPLDGFPSAAGLEALCRQDNSLPRPCPTEQLSPSHPPLITCTGSDVDVQLQVAVPQPGRYALVVEYANEDARQEVGVAVHTPQRAPQQGLLSLHPCLYSTLCRGTARDTQDHLAVFHLDSEASVRLTAEQARFFLHGVTLVPIEEFSPEFVEPRVSCISSHGAFGPNSAACLPSRFPKPPQPIILRDCQVIPLPPGLPLTHAQDLTPAMSPAGPRPRPPTAVDPDAEPTLLREPQATVVFTTHVPTLGRYAFLLHGYQPAHPTFPVEVLINAGRVWQGHANASFCPHGYGCRTLVVCEGQALLDVTHSELTVTVRVPKGRWLWLDYVLVVPENVYSFGYLREEPLDKSYDFISHCAAQGYHISPSSSSLFCRNAAASLSLFYNNGARPCGCHEVGATGPTCEPFGGQCPCHAHVIGRDCSRCATGYWGFPNCRPCDCGARLCDELTGQCICPPRTIPPDCLLCQPQTFGCHPLVGCEECNCSGPGIQELTDPTCDTDSGQCKCRPNVTGRRCDTCSPGFHGYPRCRPCDCHEAGTAPGVCDPLTGQCYCKENVQGPKCDQCSLGTFSLDAANPKGCTRCFCFGATERCRSSSYTRQEFVDMEGWVLLSTDRQVVPHERQPGTEMLRADLRHVPEAVPEAFPELYWQAPPSYLGDRVSSYGGTLRYELHSETQRGDVFVPMESRPDVVLQGNQMSITFLEPAYPTPGHVHRGQLQLVEGNFRHTETRNTVSREELMMVLASLEQLQIRALFSQISSAVFLRRVALEVASPAGQGALASNVELCLCPASYRGDSCQECAPGFYRDVKGLFLGRCVPCQCHGHSDRCLPGSGVCVDCQHNTEGAHCERCQAGFVSSRDDPSAPCVSCPCPLSVPSNNFAEGCVLRGGRTQCLCKPGYAGASCERCAPGFFGNPLVLGSSCQPCDCSGNGDPNLLFSDCDPLTGACRGCLRHTTGPRCEICAPGFYGNALLPGNCTRCDCTPCGTEACDPHSGHCLCKAGVTGRRCDRCQEGHFGFDGCGGCRPCACGPAAEGSECHPQSGQCHCRPGTMGPQCRECAPGYWGLPEQGCRRCQCPGGRCDPHTGRCNCPPGLSGERCDTCSQQHQVPVPGGPVGHSIHCEVCDHCVVLLLDDLERAGALLPAIHEQLRGINASSMAWARLHRLNASIADLQSQLRSPLGPRHETAQQLEVLEQQSTSLGQDARRLGGQAVGTRDQASQLLAGTEATLGHAKTLLAAIRAVDRTLSELMSQTGHLGLANASAPSGEQLLRTLAEVERLLWEMRARDLGAPQAAAEAELAAAQRLLARVQEQLSSLWEENQALATQTRDRLAQHEAGLMDLREALNRAVDATREAQELNSRNQERLEEALQRKQELSRDNATLQATLHAARDTLASVFRLLHSLDQAKEELERLAASLDGARTPLLQRMQTFSPAGSKLRLVEAAEAHAQQLGQLALNLSSIILDVNQDRLTQRAIEASNAYSRILQAVQAAEDAAGQALQQADHTWATVVRQGLVDRAQQLLANSTALEEAMLQEQQRLGLVWAALQGARTQLRDVRAKKDQLEAHIQAAQAMLAMDTDETSKKIAHAKAVAAEAQDTATRVQSQLQAMQENVERWQGQYEGLRGQDLGQAVLDAGHSVSTLEKTLPQLLAKLSILENRGVHNASLALSASIGRVRELIAQARGAASKVKVPMKFNGRSGVQLRTPRDLADLAAYTALKFYLQGPEPEPGQGTEDRFVMYMGSRQATGDYMGVSLRDKKVHWVYQLGEAGPAVLSIDEDIGEQFAAVSLDRTLQFGHMSVTVERQMIQETKGDTVAPGAEGLLNLRPDDFVFYVGGYPSTFTPPPLLRFPGYRGCIEMDTLNEEVVSLYNFERTFQLDTAVDRPCARSKSTGDPWLTDGSYLDGTGFARISFDSQISTTKRFEQELRLVSYSGVLFFLKQQSQFLCLAVQEGSLVLLYDFGAGLKKAVPLQPPPPLTSASKAIQVFLLGGSRKRVLVRVERATVYSVEQDNDLELADAYYLGGVPPDQLPPSLRRLFPTGGSVRGCVKGIKALGKYVDLKRLNTTGVSAGCTADLLVGRAMTFHGHGFLRLALSNVAPLTGNVYSGFGFHSAQDSALLYYRASPDGLCQVSLQQGRVSLQLLRTEVKTQAGFADGAPHYVAFYSNATGVWLYVDDQLQQMKPHRGPPPELQPQPEGPPRLLLGGLPESGTIYNFSGCISNVFVQRLLGPQRVFDLQQNLGSVNVSTGCAPALQAQTPGLGPRGLQATARKASRRSRQPARHPACMLPPHLRTTRDSYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPCILGPLEAGLFFPGSGGVITLDLPGATLPDVGLELEVRPLAVTGLIFHLGQARTPPYLQLQVTEKQVLLRADDGAGEFSTSVTRPSVLCDGQWHRLAVMKSGNVLRLEVDAQSNHTVGPLLAAAAGAPAPLYLGGLPEPMAVQPWPPAYCGCMRRLAVNRSPVAMTRSVEVHGAVGASGCPAA

SEQ ID NO: 2 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDALRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 3 - protein, 646 aa, Homo sapiens
  DOMAIN (24)..(559): Extracellular domain
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 4 - protein, 536 aa, Homo sapiens
VPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRG

SEQ ID NO: 5 - protein, 6 aa, Artificial Sequence
  Peptide linker
IEGRMD

SEQ ID NO: 6 - protein, 231 aa, Homo Sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK